(19) United States
(12) Patent Application Publication
(10) Pub. No.: US 2010/0126287 A1
(43) Pub. Date: May 27, 2010

(54) PHARMACEUTICAL ANALYSIS APPARATUS AND METHOD

(76) Inventors: Matthew D. Burke, Durham, NC (US); Lida Kalantzi, Athens (GR); Alan Frank Parr, Durham, NC (US)

Correspondence Address: GlaxoSmithKline;GLOBAL PATENTS -US, UW2220, P. O. BOX 1539, KING OF PRUSSIA, PA, 19406-0939, US

(21) Appl. No.: 12/598,476

(22) PCT Filed: May 1, 2008
(86) PCT No.: PCT/US08/62153

§ 371 Date: Nov. 2, 2009

Related U.S. Application Data
(60) Provisional application No. 60/915,995, filed on May 4, 2007.

(51) Int. Cl. G01N 33/00; G09B 23/28

(52) U.S. Cl. 73/865.6; 434/262

(57) ABSTRACT

An apparatus and method are provided for analyzing the release of active agent(s) from pharmaceutical and pharmaceutical-like products. The apparatus and method provide for more accurate simulation of the conditions in the GI tract and oral cavity including forces that are applied to the dosage form.

### FIELD OF THE INVENTION

The present invention relates to the analysis of pharmaceutical and pharmaceutical-like products. More particularly, the present invention relates to an apparatus and process for analyzing and/or predicting the release of active agents in pharmaceutical and pharmaceutical-like products.

### BACKGROUND OF THE INVENTION

Contemporary dissolution devices include a basket-type, a paddle-type and a reciprocating cylinder-type flow through device (USP IV). For example, the contemporary paddle type dissolution apparatus has a glass, round-bottomed vessel with an impeller mixing the contents of the vessel. The apparatus can also have an auto-sampler shaft inserted into the vessel to collect samples at selected intervals of time from an aqueous solution in the vessel. A tablet to be analyzed is dropped into the vessel and falls to the bottom of the vessel, where it sits during the dissolution run. The basket and reciprocating cylinder-type dissolution devices similarly provide for mixing of the solution in the device while the tablet rests in the vessel.

These contemporary dissolution devices were designed for quality control of drug release rates. The contemporary dissolution devices suffer from the drawback of failing to adequately replicate the conditions that a dosage form encounters in the gastro-intestinal (GI) tract, e.g., the stomach and/or intestine. None of these contemporary devices simulates or accounts for the forces applied to the dosage form due to the digestive conditions and peristaltic actions along the GI tract.

As shown in FIG. 1, food and liquids are present in the GI tract, in addition to mastication in the oral cavity, digestive muscular contractions, mass movement, compression, peristalsis, and other forces. All of these conditions can play a key role in the rate of drug release, especially for controlled or extended release products. These mechanically destructive forces are clearly present and are imparted on a dosage form as it travels along the GI tract.

Accordingly, there is a need for an apparatus and process for analyzing and predicting the release of active pharmaceutical ingredients (API) or active agents from pharmaceutical and pharmaceutical-like products. There is a further need for such an apparatus and process that more adequately replicates or simulates the conditions in the GI tract.

### SUMMARY OF THE INVENTION

It is an object of the present invention to provide a more accurate process and apparatus for analyzing and/or predicting release of active agents from pharmaceutical and pharmaceutical-like products.

It is another object of the present invention to provide such a process and apparatus that more adequately replicates or simulates the conditions found in the GI tract.

It is another object of the present invention to provide such a process and apparatus that more adequately replicates or simulates the conditions found in the oral cavity.

It is yet another object of the present invention to provide such a process and apparatus that more efficiently performs such analysis and/or prediction of the active agent(s) release.

These and other objects and advantages of the present invention are provided by an apparatus for analyzing the release of an active agent(s) from a pharmaceutical product or pharmaceutical-like product, which more accurately simulates the conditions in the GI tract by applying forces to the dosage form. The frequency, duration and amount of force or compression that is applied to the dosage form can be controlled and preferably varied. This is preferably done by a programmable logic computer (PLC). The analysis device is preferably retro-fitable to existing dissolution devices to render such contemporary devices more accurate in simulating the conditions in the GI tract and oral cavity.

Other and further objects, advantages and features of the present invention will be understood by reference to the following:

### BRIEF DESCRIPTION OF THE DRAWINGS

FIG. 1 is a schematic representation view of a portion of a human upper GI tract;

FIG. 2 is a plan view of an analyzing device of the present invention without an impeller and a sampler;

FIG. 3 is a perspective view of the device of FIG. 2 with the force application system actuated;

FIG. 4 is a perspective view of a portion of the device of FIG. 3;

FIG. 5 is a perspective view of the device of FIG. 2 with the impeller and the sampler;

FIG. 6 represents dissolution results for bi-layer matrix tablets over time for a contemporary USP 2 dissolution apparatus (“original dissolution”) in comparison to the deconvolution of clinical pharmacokinetics results, where the two formulations vary in the level of rate controlling polymer in the sustained release layer, which in this case was HPMC. The bilayer tablet contains an Immediate Release (IR) layer without HPMC, and a Sustained Release (SR) layer with HPMC.

FIG. 7 represents dissolution results over time for the present invention (“peristaltic dissolution”) in comparison to the deconvolution of clinical pharmacokinetics results for the bi-layer matrix tablets of FIG. 6;

FIG. 8 represents dissolution results for another sustained release dosage form over time for a contemporary USP 2 dissolution apparatus (“original dissolution”) in comparison to the deconvolution of clinical pharmacokinetics results;

FIG. 9 represents dissolution results over time for the present invention (“peristaltic dissolution”) in comparison to the deconvolution of clinical pharmacokinetics results for the dosage form of FIG. 8; and

FIG. 10 is a front elevational view of an alternative embodiment of a force application system in accordance with the invention.

### DETAILED DESCRIPTION OF THE INVENTION

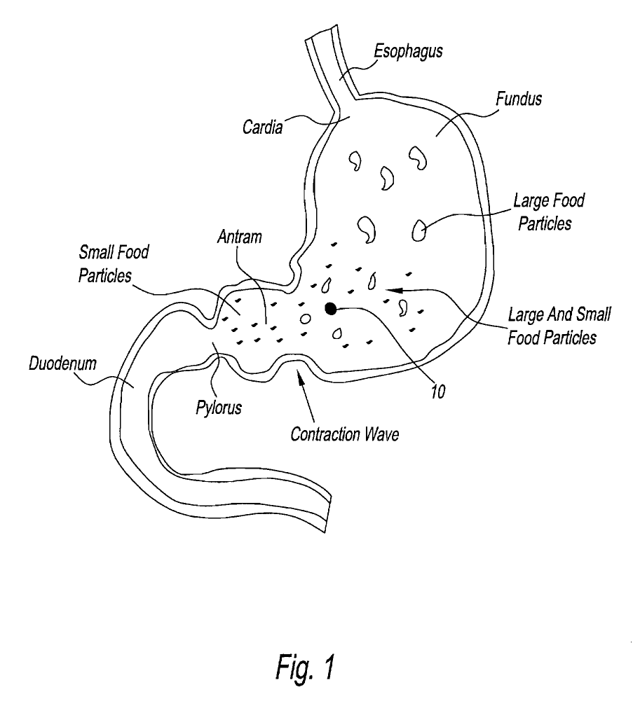

Referring to the drawings, and in particular FIG. 1, a pharmaceutical product or dosage form 10 traveling along the human GI tract is subjected to forces from a variety of sources including food and liquids that are present therein, mastication and other “oral cavity effects”, digestive muscular contractions, mass movement, compression, peristalsis, and other forces. These forces act upon the dosage form 10, effecting the release of the dosage form's active agent(s). It should be understood that while the following disclosure describes the pharmaceutical product or pharmaceutical-like product as a dosage form 10, the present invention contemplates analysis of any type of pharmaceutical product or pharmaceutical-like product that has an active agent(s) which is released, such as, for example, tablets, capsules, caplets, chewing gum, lozenges, pastilles, or other dosage forms.

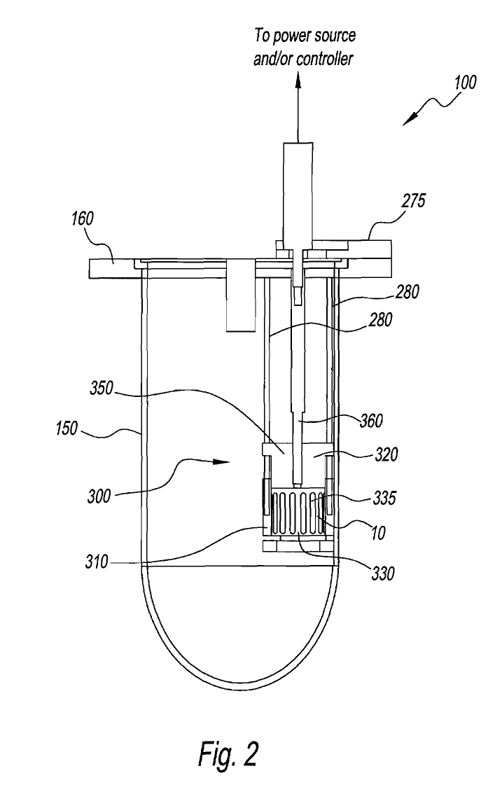

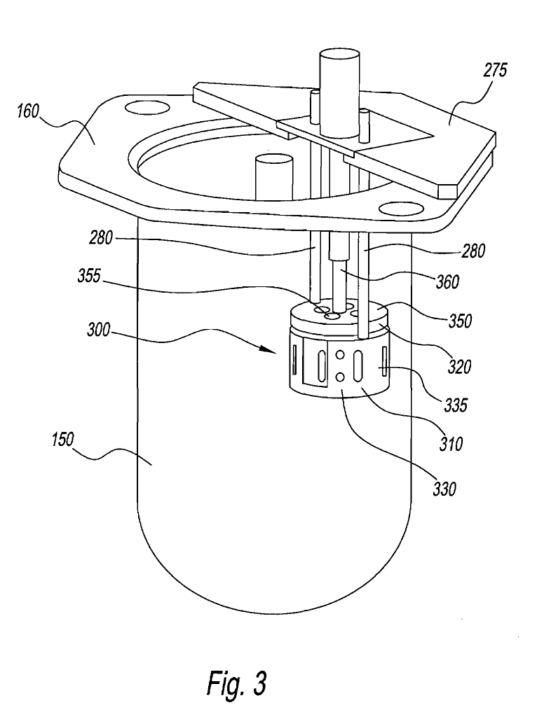

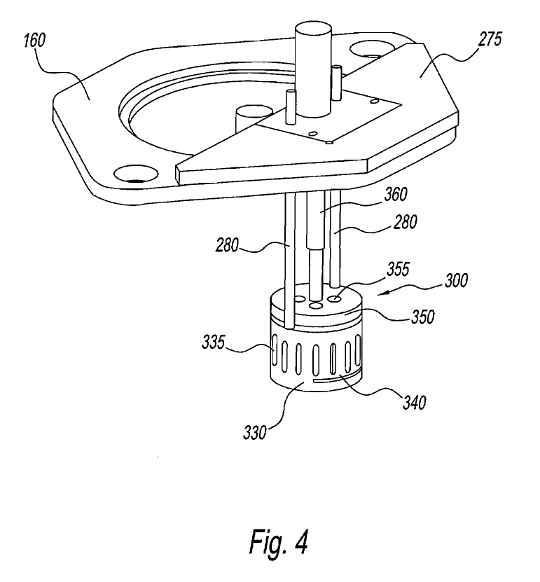

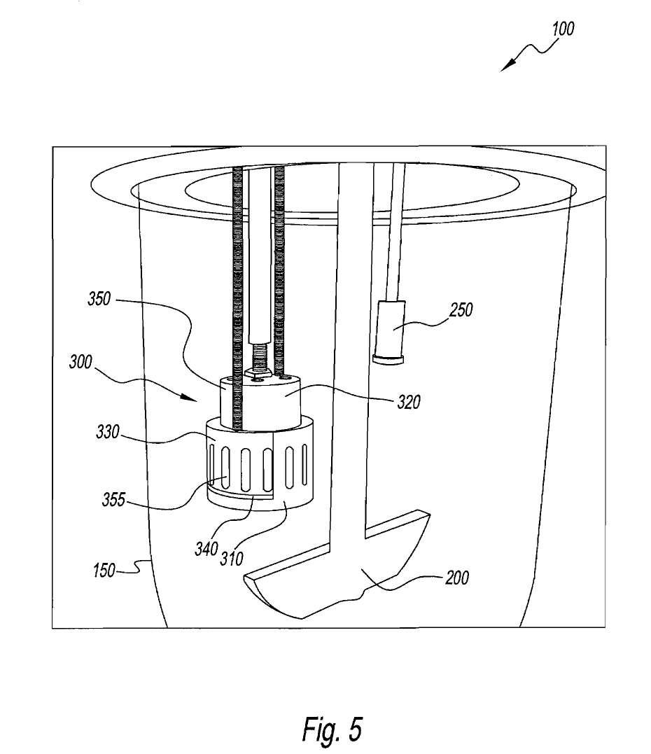

Referring to FIGS. 2 through 5, a preferred embodiment of the pharmaceutical analysis apparatus or device of the present invention is shown and generally referred to by reference numeral 100. The device 100 has a housing 150, a top 160, an impeller 200, a sampler 250, a connecting or mounting plate 275, and a force application system 300.

The housing 150 holds the solution, e.g., an aqueous solution, which simulates the medium in the human GI tract or oral cavity. The housing 150 is a transparent, round-bottomed vessel. However, the present invention contemplates the use of other materials and other shapes for the housing 150, which facilitate use of the analysis device 100 and/or more accurate simulation of the conditions of the GI tract or oral cavity.

The impeller 200 provides motion to the aqueous solution to distribute the active agent in the solution and to further simulate the conditions of the GI tract or oral cavity. The present invention contemplates the use of various shapes and sizes for the impeller 200, as well as various directions of movement for the impeller (e.g., rotational and/or axial), which can facilitate distribution of the active agent in the solution and/or more accurately simulate the conditions in the GI tract or oral cavity. The present invention also contemplates the use of other devices for distributing the active agent in the solution and for simulating the motion of the medium, solution and/or dosage form 10 in the GI tract or oral cavity, such as, for example, a reciprocating cylinder in a cylindrical vessel.

The sampler 250 obtains samples of the aqueous solution to determine the amount of active agent that has been released by the dosage form 10. Preferably, the sampler 250 is operably connected to a controller, such as, for example, a control processing unit or PLC (not shown), which can selectively obtain the sample, process it, and/or analyze it. Such analysis of the sample of the solution includes, but is not limited to, UV analysis and HPLC. However, the present invention contemplates the use of various techniques of analysis of the sample of solution.

The force application system 300 is mounted or connected with the housing 150 of the analysis device 100, and in particular with the top 160, through use of connecting plate 275. Connecting plate 275 allows for retro-fitting of the force application system 300 with a contemporary dissolution device. However, the present invention contemplates the use of other structures and methods of mounting or connecting the force application system 300 to the housing 150 or to a contemporary dissolution device. The connecting plate 275 has a number of supports 280 that allow the force application system 300 to be positioned below the connecting plate into the solution.

The present invention also contemplates the supports 280 being adjustable so that the position of the force application system 300 in the solution can be selectively varied. The present invention further contemplates the use of other structures and methods for positioning the force application system 300 in a selected position in the housing 150.

The force application system 300 has a dosage form housing 310 and a force imparting mechanism 320. In the embodiment shown, the dosage form housing 310 is a cylindrical chamber 330 having a mesh screen 340 along the bottom of the chamber. The cylindrical chamber 330 has a number of side slots 335, which allow for flow of the aqueous solution into and through the chamber. The mesh screen 340 is a floor for the chamber 330 upon which the dosage form 10 sits. Where a specific orientation of the dosage form 10 is desired, such as when analyzing a bi-layer tablet, two mesh screens 340 can be used to sandwich the dosage form in place.

In the embodiment shown, the force imparting mechanism 320 is a piston 350. The piston 350 has a number of holes 355 formed therethrough, which allow for flow of the aqueous solution into the chamber 330. The piston 350 is connected to a drive shaft 360, which can be actuated by a power source (not shown), which in this embodiment is a pneumatic cylinder. However, the present invention contemplates the use of other power sources, such as, for example, a mechanical cam or electrical solenoid, or an electric motor having a lead screw. Another device suitable for use in the force imparting mechanism is a voice coil actuator together with its associated controller. The voice coil actuator is especially desirable as it can be controlled so that it causes the plunger to move downward until it contacts the dosage form, and then stop and apply a predetermined force. In this way, as the dosage form swells, erodes, or changes dimensions during the experiments, the plunger can reliably apply the same predetermined force.

In an alternative embodiment (not shown), the force application system 300 utilizing piston 350 can have a molded surface or electropolished stainless steel or another suitable material which contacts the dosage form 10. For example, the molded surface may resemble or simulate the surface of a tooth or teeth.

In an alternative embodiment (not shown), the force application system 300 has a contact medium. The contact medium would be positioned or located on the force application system 300, where the force is imparted to the dosage form 10. For example, where force application system 300 utilizes piston 350, the contact medium could be on the piston and would make contact with the dosage form 10. The contact medium may be a silicone padding on the lower portion of piston 350 (e.g., on the ceiling of the force application system 300). The contact medium can also be a wire mesh on the lower portion of piston 350 (e.g., on the ceiling of the force application system 300).

Where the contact medium is a wire mesh, it may be assembled with various degrees of tensions (such as, for example, very tight or very loose), depending on the requirement for the dissolution method. A loose wire mesh would be used to apply the force gently on the dosage form 10, to simulate a peristaltic contraction. Wire meshes of various thicknesses of wires and various numbers of openings per square inch can be used for the contact medium.

The present invention contemplates the substantially solid piston 350 of the embodiment of FIGS. 2 through 5 being modified by attaching or connecting the contact medium, such as, for example, a perforated FDA approved silicone padding. The silicone padding can be of various thicknesses depending on the dissolution method. The use of the silicone pad mimics or simulates the environment of the GI soft tissue wall and mimics or simulates the GI peristaltic contractions.

The present invention contemplates the use of other materials and/or combinations of materials for the contact medium, which will simulate the conditions that the dosage form 10 is exposed to when in the GI tract. While this alternative embodiment has the contact medium positioned along the bottom portion of piston 350, the present invention contemplates the contact medium being located in various positions along the force application system 300, which will simulate the conditions that the dosage form 10 is exposed to when in the GI tract.

Referring back to the embodiment shown in FIGS. 2 through 5, the power source is preferably operably connected to a programmable timer or the PLC so as to automate the device 100, facilitate control of the analysis process, and allow for accurate reproduction of the analysis of dosage form 10. Force application system 300 is preferably made from electropolished stainless steel. While the dosage form housing 310 and the force imparting mechanism 320 are described in the preferred embodiment as a piston-cylinder assembly, the present invention contemplates other assemblies and devices that allow force imparting mechanism 300 to selectively apply force to the dosage form 10. Such alternative assemblies or devices preferably allow for control of the amount, duration and frequency of the compression. Additionally, such alternative assemblies also contemplate application of multiple forces and/or at varying angles to the dosage form 10 to simulate the conditions in the GI tract.

The programmable timer or PLC is used to set the time that the piston 350 stays in the down position (i.e., the compression state), the frequency at which compression occurs, and the amount of compression. The use of the PLC in conjunction with the adjustability provided by the force application system 300, allows the analysis device 100 to vary the forces (duration, frequency, amount) that are applied to the dosage form 10. The present invention also contemplates use of this controlled variation of force over the duration of the analysis to more accurately simulate the conditions that the dosage form is subjected to as it travels along the GI tract.

Cylindrical chamber 330 preferably has an outer diameter of about 32 mm, an inner diameter of about 24 mm, and a height of about 26 mm. The side slots 335 in cylindrical chamber 330 preferably are about 14 mm in height and about 2.6 mm in width. To hold the mesh screen 340 in place in the cylindrical chamber 330, there are two cuts in the lower part of the chamber that are preferably about 22 mm in width and 1.5 mm in height, so that the screen material can be inserted therein.

The cylindrical chamber 330 is preferably located about 8 cm below the connecting plate 275. The piston 350 preferably has an outer diameter of about 23.5 mm and a height of about 19 mm. The piston 350 has four holes 355 drilled axially through the piston that preferably each have a diameter of about 6.3 mm to allow for the fluid flow therethrough. While this embodiment uses the above described dimensions to simulate the conditions in a human GI tract, the present invention contemplates the use of other dimensions to facilitate control of the analysis process and allow for accurate reproduction of the analysis of dosage form 10.

The present invention contemplates the use of other materials for the mesh screen 340 such as stainless steel or suitable plastics, such as those used in the traditional USP 3 dissolution apparatus. The mesh size of the mesh screen 340 can also be varied as appropriate for the particular dosage form 10.

The pneumatic cylinder, which provides for the motion of the piston 350, is connected to the programmable timer or PLC via two tubes (not shown) and a compressed air source is connected to the programmable timer with a regulator (not shown) connected to adjust the air pressure. The regulator can be used to control the force that is imparted upon the dosage form 10 via regulating the amount of air pressure. As the piston 350 moves to the lower position, it compresses the dosage form 10 against the mesh screen 340 thus applying a mechanical stress to the dosage form 10 simulating the in-vivo forces that the dosage form would experience.

In a further embodiment of the invention, dosage forms such as medicated chewing gums which are retained in the oral cavity and release the active ingredient into the mouth, may also have a need for dissolution methodology that can mimic chewing frequency and intensity. One class of drug substances, e.g. lipophilic agents, may dissolve in the saliva-insoluble gum base and thereafter only be slowly released during mastication.

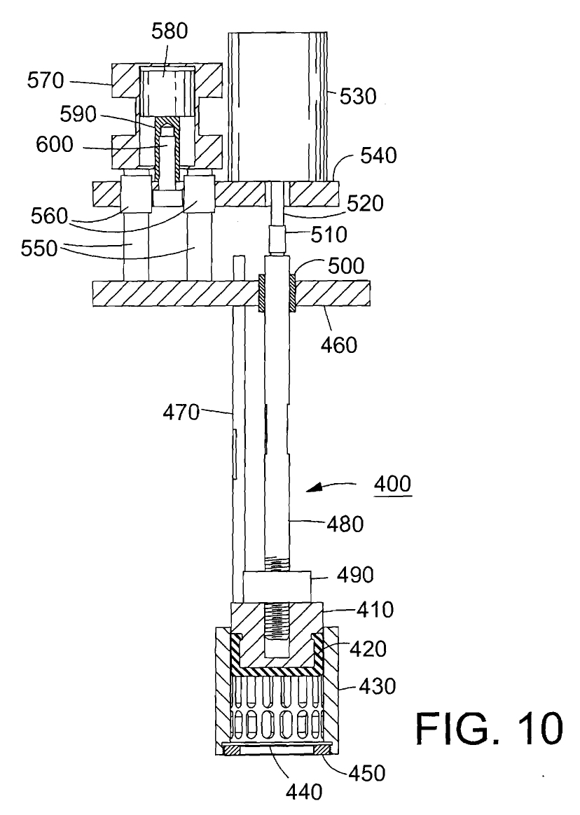

In the force application system 400, illustrated in FIG. 10, a piston 410, having a silicone piston cap 420, is both vertically reciprocable, and rotatable, in a foraminous, cage-like, dosage form chamber 430, which is similar to chamber 330 in FIG. 2. The chamber is provided with a wire mesh screen 440 and a screen retainer 450, and is supported from a fixed platform 460 by a pair of rods, one of which is rod 470. The other support rod is not shown because it is in front of the section plane.

The piston 410 is threaded onto the threaded lower end of a piston rod 480, and secured by a threaded clamp 490. The rod 480 extends through a guide bushing 500 in platform 460, in which the rod is both vertically slidable and rotatable, and is coupled, by means of a shaft coupling 510, to the shaft 520 of a motor 530. The motor can be an electric motor having suitable internal reduction gearing, a pneumatically or hydraulically operated rotary actuator, or any other form of motor suitable to impart a predictable, and preferably controllable, rotation to the piston 420 by rotating rod 480.

Motor 530 is mounted on a movable platform 540, which has a hole through which the motor shaft 520 extends. Platform 540 is guided for vertical reciprocatory movement on a pair of guide rods 550, which are fixed to platform 460 and extend through bushings 560 mounted in the movable platform. Optionally, one or more additional guide rods can be provided.

Guide rods 550 also support a linear actuator 570, which can be a pneumatic or hydraulic actuator having an internal piston 580, as shown. Alternatively, the actuator 570 can be an electric motor having a lead screw, or any other suitable form of linear actuator capable of applying a predictable, and preferably controllable, force. The actuator shaft 590 is connected to movable platform 540 by a fastener 600.

In operation, the actuator 570 can effect vertical reciprocation of platform 540, which, in turn, effects linear reciprocation of piston 410 through rod 480 in opposite directions. Simultaneously or alternatively, motor 530 can be operated to rotate the piston about an axis parallel to the directions of reciprocation. That is, the piston can be both rotated and reciprocated at the same time in order to simulate chewing, or it can be rotated while the piston is held at a fixed height.

The force application system 400 is capable of performing the same function as that of the force application system 300 described above. That is, by causing the piston 410 to move linearly, the system can be made to simulate conditions in a patient's gastrointestinal tract. In addition, however, the force application system is capable of imparting rotation to the piston, either with or without simultaneous linear movement, in order to simulate conditions in a patient's oral cavity. Thus, the apparatus of FIG. 10 permits not only study of the dissolution of an oral dosage form in the GI tract, but also study of dissolution of the dosage form during the process of mastication.

The conditions of operation of the apparatus, including the amount and rate of linear movement of the piston, the amount and rate of rotation of the piston, the repetition rate of reciprocatory movement and rotation can all be controlled electronically by control systems well known in the art. Although the controls can operate in a feed-forward mode, feedback can be introduced by incorporation of strain gauges or other suitable measuring devices into rod 480.

In a typical mode of operation, it is contemplated that the piston will be caused to rotate through a half turn, a full turn, or more, while moving to its lowermost position. This action enables the piston to simulate grinding as well as a compressive action on the dosage form. This mode can be utilized to simulate the conditions in the oral cavity for chewing gum formulations.

Various modifications to the dual mode apparatus of FIG. 10 can be made. For example, the turning action of the piston can be achieved by having the piston rotate on the piston rod, while cooperating projections and helical cam grooves associated with the piston and the housing automatically cause the piston to rotate by a predetermined amount as it approaches the bottom of the housing. Alternatively, other mechanisms which would result in the piston rotating during compression could be designed by those skilled in the art.

The device 100 is flexible in its settings and sizes. The materials used for force application system are those that are able to withstand prolonged exposure to acid and to basic pH with and/or without various surfactants commonly used in pharmaceutical dissolution analysis. However, it has been found that certain materials are not properly suited for the process described above. Materials that have been found to be inadequate for these purposes are untreated stainless steel, thinly coated PTFE stainless steel, and hard anodized stainless steel. Such materials corroded after a series of experiments when using acid pH dissolution media. One such material that was found to be usable in the above-described apparatus was electropolished stainless steel.

The overall dimensions of the device 100 are dictated in part by the size of the vessel or housing 150, the size of the impeller 200, the size of the impeller shaft and location, the size of the sampler tube 250, and any filter being used. The maximum diameter of the chamber 330 or 430 and piston 350 or 410 would preferably be the size that fits into the housing 150 but does not contact the side of the housing, impeller 200 and sampler 250. Preferably, the maximum internal diameter of the chamber and the outer diameter of the piston are only as large as the maximum size that the formulation analyzed achieves. However, this maximum size can be fairly large when considering large swelling shapes for gastric retention. When evaluating expanding gastric retentive dosage forms, the mesh screen 340 or 440 can be replaced by a component similar in shape to a funnel with a fixed or modulated opening of a size similar to a pyloric sphincter. By recording the time the formulation is retained in the chamber, one can predict when gastric emptying of the dosage form will occur in-vivo.

Where the components of device 100 are retro-fitted to a USP 2 paddle-type dissolution apparatus, the device is able to utilize all of the benefits of the traditional USP 2 apparatus, and add an advantage of the ability to hold the dosage form 10 in a piston type device (force application system 300 or 400) that is able to apply physical force to the dosage form periodically to simulate the in-vivo forces that the dosage form will be exposed to. The targeted types of dosage forms that will benefit more from this analysis are, for the most part, controlled or extended release products. However, the present invention contemplates the use of this apparatus and method on all types of pharmaceutical products including immediate release dosage forms.

It should be understood that the apparatus and method described herein has been discussed with respect to simulating the conditions in the human GI tract and oral cavity. However, the present invention contemplates the use of the apparatus and method for simulation of other GI tracts and oral cavities where applicable.

In another alternative embodiment (not shown), force application system 300 has a bag or pouch to hold the dosage form 10. Preferably, the bag is made from a wire mesh cloth. The wire mesh cloth is preferably woven and would use an appropriate gauge of wire with a suitable opening size. The bag would abut, or be in proximity to, a piston that is preferably operably connected to the housing 150. The dosage form 10 would be placed in the bag and the bag would be squeezed via the piston so that there would be a gentle force applied to the dosage form 10 by the squeezing motion of the wire mesh bag. This alternative structure and method for applying a force to dosage form 10 via force application system 300 would simulate or mimic the peristaltic contraction of the GI tract.

A similar modification can be made to the force application system 400, which is capable of rotation as well as reciprocation.

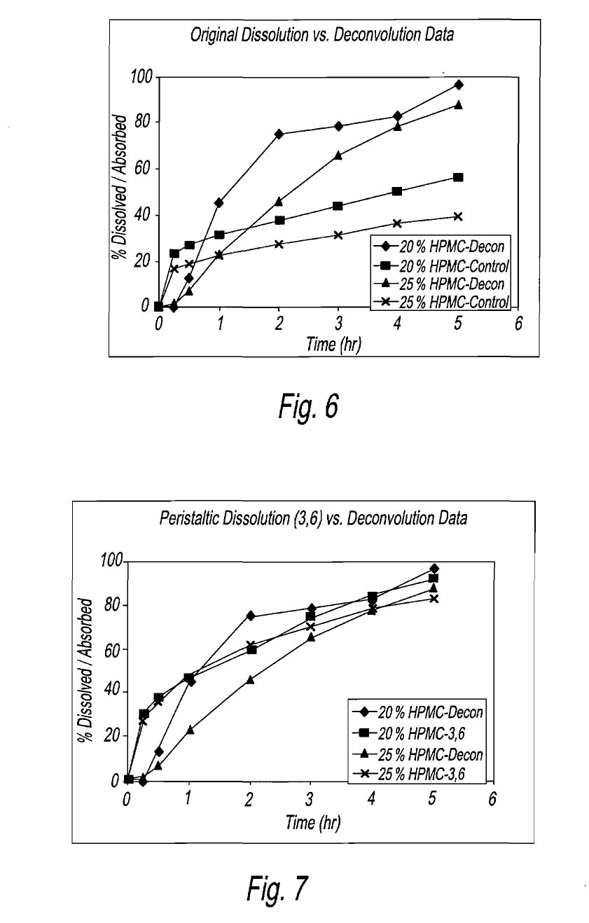

Referring to FIGS. 6 and 7, a graphical comparison is provided, which is indicative of the improved accuracy of the analysis device 100 as compared to a contemporary paddle-type USP 2 dissolution device for predicting dissolution of bi-layer matrix tablets. The dissolution for the contemporary USP 2 dissolution apparatus (“original dissolution”) of FIG. 6 and the dissolution for the device 100 (“peristaltic dissolution”) of FIG. 7 are shown in comparison to the deconvolution of clinical pharmacokinetics results for the bi-layer matrix tablet.

For the results shown in FIG. 7, the force application system 300 of device 100 utilized a compression time of three seconds with six seconds in between compressions (i.e., “3,6”). The force was applied using air pressure at 3 bars. The accuracy of device 100 is especially evident over longer periods of time, e.g., release occurring after one hour. The apparatus and method of the present invention provides for more accurate prediction of release and, in particular, sustained release, of the active agent(s). Such accuracy and reliability in predicting release performance may allow for the reduction of the number of clinical studies required of a particular pharmaceutical product, when analyzed by the apparatus and method of the present invention.

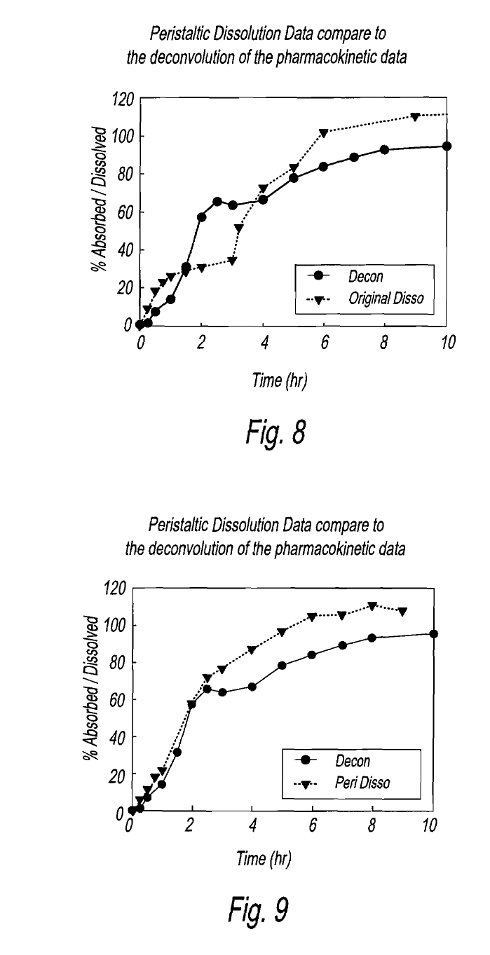

Referring to FIGS. 8 and 9, another graphical comparison is provided, which is again indicative of the improved accuracy of the analysis device 100 as compared to a contemporary paddle-type USP 2 dissolution device for predicting dissolution but of another type of dosage form. The dissolution for the contemporary USP 2 dissolution apparatus (“original dissolution”) of FIG. 8 and the dissolution for the device 100 (“peristaltic dissolution”) of FIG. 9 are shown in comparison to the deconvolution of clinical pharmacokinetics results for the dosage form.

Device 100 has been described as a single analyzing unit. However, the present invention contemplates the use of a number of devices 100, which can be used for analysis of the dosage form 10. In one such alternative embodiment, there are six devices 100 with each having a force application system 300 that are connected to one another via a common plate, rack or other structure. Such an arrangement allows for simultaneous analysis of a plurality of dosage forms 10 where the force application systems 300 are lowered together into their respective dissolution media (in their respective housings 150) at the beginning of the dissolution run. This alternative embodiment also allows for the use of coordinated control to make the process more efficient.

While the present invention has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments as described herein and in the claims.

What is claimed is:

1. A dissolution device to analyze the release of an active agent from a dosage form in a GI tract, the device comprising:
a vessel having an open end, said vessel containing a medium; and
a force application system having a chamber and a force imparting mechanism, said chamber supporting the dosage form and being in fluid communication with said medium, wherein said force imparting mechanism applies compression to the dosage form to simulate conditions in the GI tract.

2. The device of claim 1, wherein said force application system is connected to said open end thereby being positioned in said medium.

3. The device of claim 1, wherein said chamber is a cylinder, said force imparting mechanism comprises a piston, and said piston is operably connected to a power source.

4. The device of claim 1, wherein said force application system has padding positioned between the dosage form and said force imparting mechanism.

5. The device of claim 4, wherein said padding is silicon.

6. The device of claim 3, further comprising a controller operably connected to said piston to control at least one of a frequency, a duration or an amount of said compression that is applied to the dosage form.

7. The device of claim 3, further comprising an impeller that circulates said medium.

8. The device of claim 7, further comprising a sampler that obtains a sample of said medium for analysis.

9. The device of claim 3, wherein said cylinder has one or more slots that allow for flow of said medium into and through said cylinder.

10. The device of claim 9, wherein said cylinder has a mesh screen along a bottom of said cylinder.

11. The device of claim 3, wherein said cylinder has a first mesh screen along a bottom of said cylinder and a second mesh screen above said first mesh screen that sandwiches the dosage form in place.

12. The device of claim 1, wherein said force application system is made at least in part from electropolished stainless steel.

13. A method of analyzing the release of an active agent from a dosage form in a GI tract, the method comprising:
positioning the dosage form in a medium;
circulating the medium;
applying compression to the dosage form to simulate conditions in the GI tract; and
collecting data representative of the release of the active agent from the dosage form.

14. The method of claim 13, further comprising controlling a frequency of said compression that is applied to the dosage form.

15. The method of claim 13, further comprising controlling a duration of said compression that is applied to the dosage form.

16. The method of claim 13, further comprising controlling an amount of said compression that is applied to the dosage form.

17. The method of claim 13, wherein the dosage form is positioned in a bag that is squeezed for applying compression to the dosage form.

18. The method of claim 13, further comprising collecting second data representative of the time of gastric emptying of the dosage form in-vivo.

19. The method of claim 18, wherein said second data is collected by monitoring the time the dosage form is retained in a chamber having a fixed or modulated opening of a size similar to a pyloric sphincter.

20. A dissolution device to analyze the release of an active agent from an oral dosage form, the device comprising:
a vessel for containing a fluid medium; and
a force application system comprising a chamber for supporting the dosage form, the chamber having at least one passage for fluid communication with said medium in the vessel, and also comprising a piston, movable within the chamber, for applying compression to the dosage form.

21. A dissolution device according to claim 20, in which said piston is reciprocable in said chamber in opposite directions toward and away from a dosage form therein.

22. A dissolution device according to claim 21, in which said piston is also rotatable in said chamber about an axis parallel to said directions.

23. A dissolution device according to claim 22, in which said piston is simultaneously reciprocable in said opposite directions and rotatable about said axis.

24. A dissolution device according to claim 22, including a first actuator for causing reciprocation of said piston, and a second actuator for causing rotation of said piston.

25. A method of analyzing the release of an active agent from a dosage form, the method comprising:
positioning the dosage form in a fluid medium;
circulating the medium;
while applying compression to the dosage form by means of a pressure-applying piston, rotating the piston to effect a grinding action on the dosage form; and
collecting data representative of the release of the active agent from the dosage form.

26. A dissolution device to analyze the release of an active agent from a dosage form in the oral cavity, the device comprising:
a vessel having an open end, said vessel containing a medium; and
a force application system having a chamber and a force imparting mechanism, said chamber supporting the dosage form and being in fluid communication with said medium, wherein said force imparting mechanism applies compression to the dosage form while rotating to simulate grinding as well as compression conditions in the oral cavity.

27. The device according to claim 26 wherein the rate and intensity of the compression force is varied.

28. The device according to claim 26 wherein the rate and intensity of the grinding force is varied.
